(19) United States
(12) Patent Application Publication
(10) Pub. No.: US 2026/0054097 A1
(43) Pub. Date: Feb. 26, 2026

(54) METHODS AND SYSTEMS FOR MEASURING AND CONTROLLING RADIOSURGERY SYSTEMS

(71) Applicant: Ohio State Innovation Foundation, Columbus, OH (US)

(72) Inventors: J. MATHEWS, Tacoma, WA (US); Lanchun LU, Columbus, OH (US); Matthew MEINEKE, Columbus, OH (US); Nilendu GUPTA, Columbus, OH (US)

(21) Appl. No.: 19/107,370

(22) PCT Filed: Aug. 30, 2023
(86) PCT No.: PCT/US2023/031553

§ 371 Date: Feb. 27, 2025

Related U.S. Application Data
(60) Provisional application No. 63/402,120, filed on Aug. 30, 2022.

(51) Int. Cl. A61N 5/10

(52) U.S. Cl. CPC A61N 5/1075; A61N 5/1078; A61N 5/1084

(57) ABSTRACT

An example quality assurance (QA) system for a radiosurgery system is described herein. The system includes an appliance comprising: a housing, an indicator; and an actuator configured to move the indicator; a controller in operable communication with the appliance, where the controller comprises a processor and a memory, the memory having computer-executable instructions stored thereon that, when executed by the processor, cause the processor to: transmit one or more control signals to the appliance in accordance with a QA test protocol, where the QA test protocol comprises moving the actuator from a first position to a second position, the first and second positions being separated by at least a first predetermined distance.

### CROSS-REFERENCE TO RELATED APPLICATIONS

This application claims the benefit of U.S. provisional patent application No. 63/402,120, filed on Aug. 30, 2022, and titled “METHODS AND SYSTEMS FOR MEASURING AND CONTROLLING RADIOSURGERY SYSTEMS,” the disclosure of which is expressly incorporated herein by reference in its entirety.

### BACKGROUND

Radiosurgery systems can treat target areas of the body using focused beams of radiation. The radiation can cause selective ionization of tissue, destroying cells. Radiosurgery can be used to, for example, treat cancer by ionizing cancer cells in specific areas of the body. Because radiosurgery systems can destroy all cells, not just cancer cells, it is important that radiosurgery systems are reliable and accurate. Thus, there is a need for methods and systems for improving and/or verifying the reliability and/or accuracy of radiosurgery systems.

### SUMMARY

Methods and systems for performing quality assurance of a radiosurgery system are described herein.

In some aspects, the techniques described herein relate to a quality assurance (QA) system for a radiosurgery system, the system including: an appliance including: a housing, an indicator; and an actuator configured to move the indicator; a controller in operable communication with the appliance, wherein the controller includes a processor and a memory, the memory having computer-executable instructions stored thereon that, when executed by the processor, cause the processor to: transmit one or more control signals to the appliance in accordance with a QA test protocol, wherein the QA test protocol includes moving the actuator from a first position to a second position, the first and second positions being separated by at least a first predetermined distance.

In some aspects, the techniques described herein relate to a system, wherein the QA test protocol further includes repeatedly moving the actuator between the first and second positions a predetermined number of times.

In some aspects, the techniques described herein relate to a system or claim 2, wherein the QA test protocol further includes maintaining the actuator in the second position for a predetermined period of time.

In some aspects, the techniques described herein relate to a system, further including a communication link, wherein the communication link operably connects the controller and the appliance.

In some aspects, the techniques described herein relate to a system, wherein the communication link is a wired communication link.

In some aspects, the techniques described herein relate to a system, wherein the communication link is a wireless communication link.

In some aspects, the techniques described herein relate to a system, wherein the one or more control signals are configured to move the actuator a second predetermined distance.

In some aspects, the techniques described herein relate to a system, wherein the appliance further includes a sensor configured to detect radiation.

In some aspects, the techniques described herein relate to a system, wherein the sensor is a photodiode.

In some aspects, the techniques described herein relate to a system, wherein the sensor is a ionization detector, scintillation detector or semiconductor detector.

In some aspects, the techniques described herein relate to a system, wherein the memory has further computer executable instructions stored thereon that, when executed by the processor, cause the processor to: detect, using the sensor, a radiation emitted from the radiosurgery system; while the radiation is detected by the sensor, send the one or more control signals to the appliance in accordance with the QA test protocol at a first time; detect, using the sensor, that the radiosurgery system is no longer emitting the radiation at a second time; and output a time difference between the first time and the second time.

In some aspects, the techniques described herein relate to a system, further including a fiducial marker.

In some aspects, the techniques described herein relate to a system, wherein the fiducial marker is a removable fiducial marker.

In some aspects, the techniques described herein relate to a system, further including a plurality of fiducial markers including a first fiducial marker and a second fiducial marker.

In some aspects, the techniques described herein relate to a system, wherein the first and second fiducial markers are offset from one another by a predetermined orientation and a predetermined position.

In some aspects, the techniques described herein relate to a system or claim 15, wherein the first fiducial marker is a removable fiducial marker.

In some aspects, the techniques described herein relate to a system, wherein the memory has further computer executable instructions stored thereon that, when executed by the processor, cause the processor to: receive location information from the radiosurgery system, the location information including an estimate of the position of the first fiducial marker and an estimate of the position of the second fiducial marker, and determine, based on the location information, an accuracy value for the radiosurgery system.

In some aspects, the techniques described herein relate to a system, wherein the appliance is operably coupled to the radiosurgery system.

In some aspects, the techniques described herein relate to a system, wherein the memory has further computer executable instructions stored thereon that, when executed by the processor, cause the processor to: receive an instruction from the radiosurgery system; and control the actuator in response to the received instruction.

In some aspects, the techniques described herein relate to a system, wherein the memory has further computer executable instructions stored thereon that, when executed by the processor, cause the processor to: transmit calibration information to the radiosurgery system.

In some aspects, the techniques described herein relate to a system, wherein the actuator is a linear actuator.

In some aspects, the techniques described herein relate to a system, wherein the actuator is a stepper motor.

In some aspects, the techniques described herein relate to a system, wherein the radiosurgery system includes a linear accelerator (LINAC).

In some aspects, the techniques described herein relate to a system, wherein the radiosurgery system is a gamma knife system.

In some aspects, the techniques described herein relate to a system, wherein the indicator includes a reflector.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1A illustrates a perspective view of a schematic of a quality assurance appliance that can be part of a quality assurance system for a radiosurgery system, according to one implementation of the present disclosure.

FIG. 1B illustrates a top view of quality assurance appliance that can be part of a quality assurance system illustrated in FIG. 1A.

FIG. 1C illustrates an exploded view of quality assurance appliance that can be part of a quality assurance system illustrated in FIGS. 1A and 1B.

FIG. 2 illustrates a flowchart of a method for performing quality assurance of a radiosurgery system, according to one implementation of the present disclosure.

FIG. 3A illustrates a perspective view of an example quality assurance system for a radiosurgery system, according to one implementation of the present disclosure.

FIG. 3B illustrates an example controller for the example quality assurance system illustrated in FIG. 3A.

FIG. 4 illustrates a radiosurgery system including the example quality assurance system illustrated in FIG. 3A.

FIG. 5A illustrates test results where a patient has deviated from the treatment position a sixth time, after repeatedly deviated from the treatment position beyond a tolerance value for five times.

FIG. 5B illustrates test results where the patient has deviated beyond the tolerance from the treatment position continuously for more than 25 seconds.

FIG. 6 is an example computing device.

### DETAILED DESCRIPTION

Unless defined otherwise, all technical and scientific terms used herein have the same meaning as commonly understood by one of ordinary skill in the art.

Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for quality assurance of a gamma knife, it will become evident to those skilled in the art that the implementations are not limited thereto, but are applicable for performing quality assurance and/or control of any radiosurgery system.

Described herein are systems and methods for performing quality assurance and control of radiosurgery systems. Non-limiting examples of radiosurgery systems include linear accelerators (LINAC) and gamma knife systems.

A gamma knife can include a High-Definition Motion Management System (HDMMS), that can be a is a key device in the gamma knife radiosurgery system to monitor patient's motion during mask-based gamma knife radiosurgery. The HDMM system can have a manufacturer built-in feature-during treatment, if the patient position deviates from the planned treatment position, and the magnitude of deviation is beyond the preset tolerance, the radiation beam will be automatically turned off until the patient moves back to the treatment position within the tolerance, and then the radiation beam resumes. This feature of a HDMM is important to the radiosurgery system because it prevents healthy tissue of a patient from being harmed by the patient moving during treatment. Thus, it can be important to have systems and methods in place to validate that the HDMM is functioning correctly to prevent the user from moving during treatment and having healthy tissue ionized by radiation.

As a non-limiting example, to provide context, the following rules can be applied by an example HDMMS:

In any of the following two circumstances the Gamma Knife machine will automatically pause the treatment and move the patient outside the Gamma Knife treatment machine to check the cause of deviation or to acquire a new set of onboard Cone Beam CT (CBCT) images to check and redefine the patient's position: (1) if the patient has repeatedly deviated from the treatment position and beyond the tolerance of five times and at the sixth time, or (2) if the patient has deviated beyond the tolerance from the treatment position continuously for more than 25 seconds. Performing routinely QA check on this manufacture built-in feature to make sure the HDMM system functions properly for patient treatments is a regulation requirement. Again, it should be understood that these rules for treatment using a gamma knife are intended only as non-limiting examples to provide context into an example use case of the present disclosure.

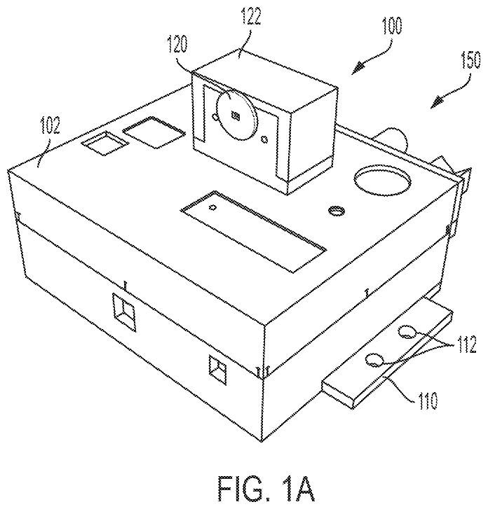

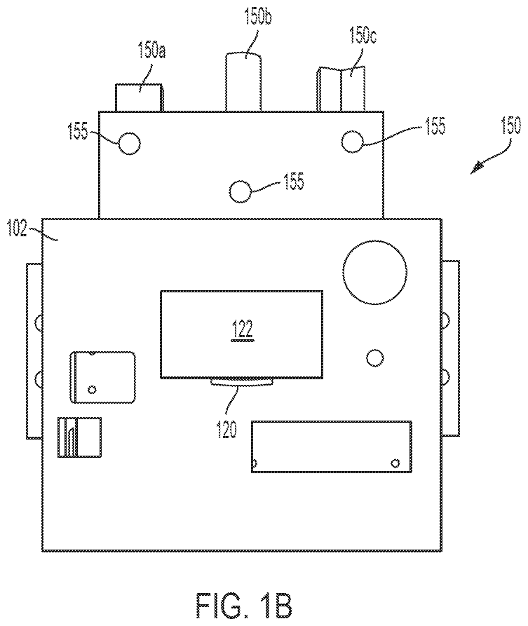

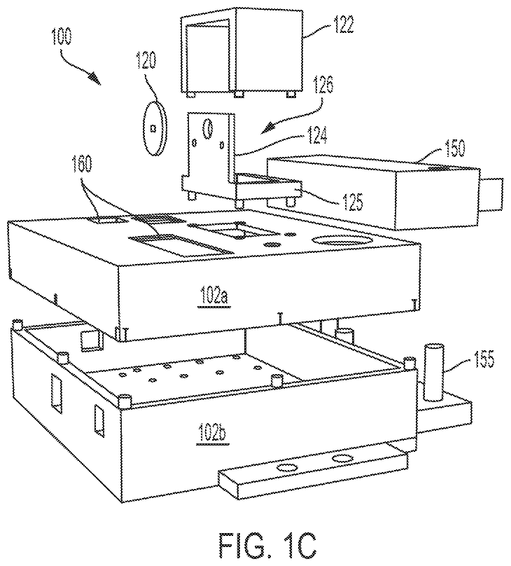

FIG. 1A illustrates a perspective view of a schematic of a quality assurance appliance 100 for a radiosurgery system, according to one implementation of the present disclosure.

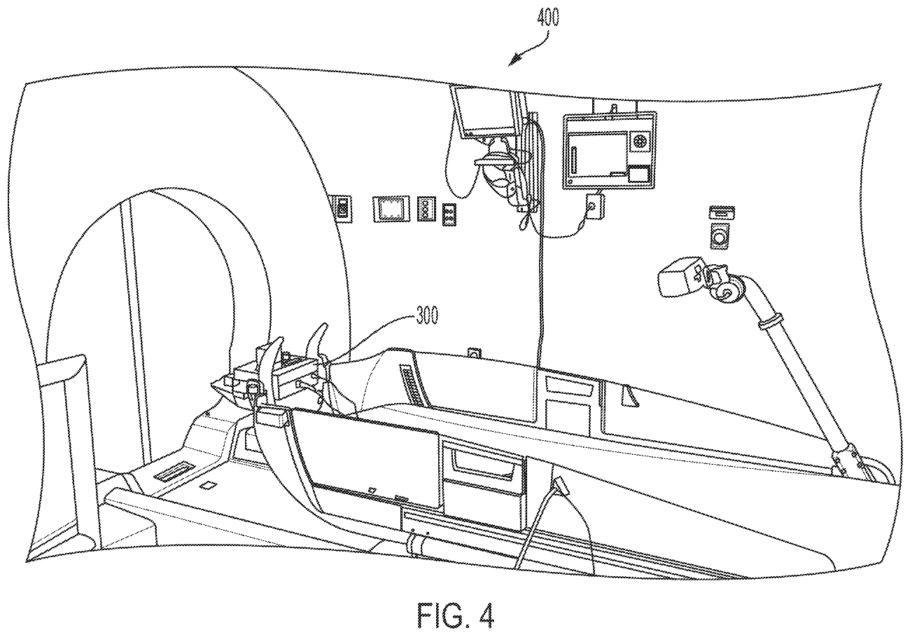

The appliance 100 can include a housing 102. The housing 102 can include one or more mounting brackets 110 with holes 112 configured to attach the housing 102 to a radiosurgery system (not shown). A non-limiting example of a radiosurgery system is the gamma knife system 400 shown in FIG. 4 and described herein with an example appliance 300, according to implementations of the present disclosure. It should be understood that different radiosurgery systems can include different mounting systems, and that implementations of the present disclosure can include different arrangements of brackets 110, holes 112, or different mounting systems.

The appliance 100 can also include an indicator 120. The indicator 120 can be mounted on a protrusion 122 from the housing that offsets the indicator 120 by a known amount from the housing 102. In some implementations, the HDMM system can be configured to measure/detect movement of a patient by tracking the movement of one or more indicators attached to the patient (e.g. to the patient's scalp). Optionally, the indicator is a reflector that can be tracked by a camera that can be part of the HDMM system.

The housing 102 and/or protrusion 122 can include an actuator (shown in FIG. 1C) operably connected to the indicator to move the indicator.

The housing 102 can also include one or more fiducial markers 150.

FIG. 1B illustrates a top view of the quality assurance appliance 100 showing the protrusion 122, indicator 120, and the fiducial marker 150. The fiducial marker 150 can be removable. As shown in FIG. 1B, the fiducial marker 150 can be attached to the housing 102 by one or more rods 155. The rods can align the fiducial marker in a known position relative to the housing 102.

The fiducial marker 150 can include one or more shapes 150a 150b 150c that act as individual fiducial markers. It should also be understood that any number of fiducial markers can be attached to the housing 102. For example, by stacking the fiducial markers 150 on the rods 155, or attaching different fiducial markers 150 to different rods 155. The relative position and orientation of the fiducial markers 150 can be predetermined, and stored in the memory 604 of one or more computing devices.

The fiducial marker 150 can be adapted to measure the accuracy of a radiosurgery system. The appliance 100 can be operably coupled to the radiosurgery system. The radiosurgery system can estimate a distance between one or more fiducial markers of the appliance, and the appliance can compare the estimate to the known distance between the fiducial markers. The accuracy of the radiosurgery system can be determined by comparing the distance estimated by the radiosurgery system and the known distance between the fiducial markers.

Alternatively or additionally, the controller can be operably coupled to the radiosurgery system. The radiosurgery system can estimate a distance between one or more fiducial markers of the appliance, and the controller can compare the estimate to the known distance between the fiducial markers. The accuracy of the radiosurgery system can optionally be determined by the controller by comparing the distance estimated by the radiosurgery system and the known distance between the fiducial markers.

The appliance 100 and/or the controller discussed above can be coupled to the radiosurgery system through one or more communication links. Alternatively or additionally, the appliance 100 and the controller can be coupled to one another through one or more communication links. This disclosure contemplates the communication links are any suitable communication link. For example, a communication link may be implemented by any medium that facilitates data exchange including, but not limited to, wired, wireless and optical links.

It should also be understood that the controller and the appliance 100 can be operably coupled in some implementations of the present disclosure. The communication link or links between the controller and the appliance 100 can be in addition to communication links between the radiosurgery system and the controller, and/or the radiosurgery system and the appliance 100. For example, in some implementations, the controller can be operably coupled to the appliance 100 and also to the radiosurgery system. The controller can optionally send and receive information from appliance 100 and can also optionally send and receive information from the radiosurgery system. In some implementations, the controller can control the actuator 124 and/or the radiosurgery system by sending control signals over the communication links. As another example, the controller can be configured to calibrate the radiosurgery system by sending control signals over the communication links, the control signals causing the actuator 124 of the appliance 100 to move the indicator 120 of the appliance 100, receiving a measurement of the position of the indicator 120 from the radiosurgery system over the communication links, and calibrating the radiosurgery system based on the actuator's position and measured position of the indicator 120 (which is moved by the actuator 124) received from the radiosurgery system. The calibration can be performed by transmitting calibration information to the radiosurgery system by sending exchanging information over the communications links. For example, the calibration information can be based on the difference between the position of the indicator 120 of the appliance 100, and the detected position of the indicator 120 measured at the radiosurgery system.

The appliance 100 can also be configured to be controlled by the radiosurgery system. For example, the radiosurgery system can be configured to move the actuator 124 and/or indicator 120 predetermined amounts to determine if the radiosurgery system is correctly calibrated or configured, as well as to determine calibration information that can be used to calibrate the radiosurgery system. The appliance 100 can be configured to transmit calibration information to the radiosurgery system.

As shown in FIG. 1C, the appliance 100 can be made of any number of individual pieces, which can be fabricated separately (for example, by 3D printing). For example, the housing 102 can include a first half 102a and a second half 102b.

The appliance 100 can also include a controller (not shown) operably connected to the actuator 124 to move the indicator 120. Non-limiting examples of actuators 124 that can be used in implementations of the present disclosure include linear actuators and stepper motors. Optionally the actuator 124 can be positioned on a frame 125, where the frame 125 can orient that actuator 124 and the indicator 120. The frame 125 can be positioned on the housing (for example on the first half 102a of the housing 102, as shown in FIG. 1C).

In some implementations of the present disclosure, the actuator 124 can include a shaft 126, as shown in FIG. 1C. The shaft 126 can couple the actuator 124 to the indicator 120. For example, if the actuator 124 is a linear motor or a stepper motor, the motor can be configured to drive the shaft 126 so that the actuator 124 moves the indicator 120 by the shaft. Additionally, it should be understood that in some implementations, the shaft 126 can be any linkage with any number of parts that couple the actuator 124 to the indicator 120.

As shown in FIG. 1C, the quality assurance appliance 100 can also include one or more sensors 160. Optionally, the sensors 160 can be radiation sensors configured to detect radiation. The sensors 160 can be operably connected to the controller (not shown). Non-limiting examples of sensors that can be used in implementations of the present disclosure include a photodiode, an ionization detector, a scintillation detector and a semiconductor detector. It should be understood that the number and/or arrangement of sensors 160 shown in FIG. 1C is provided only as an example.

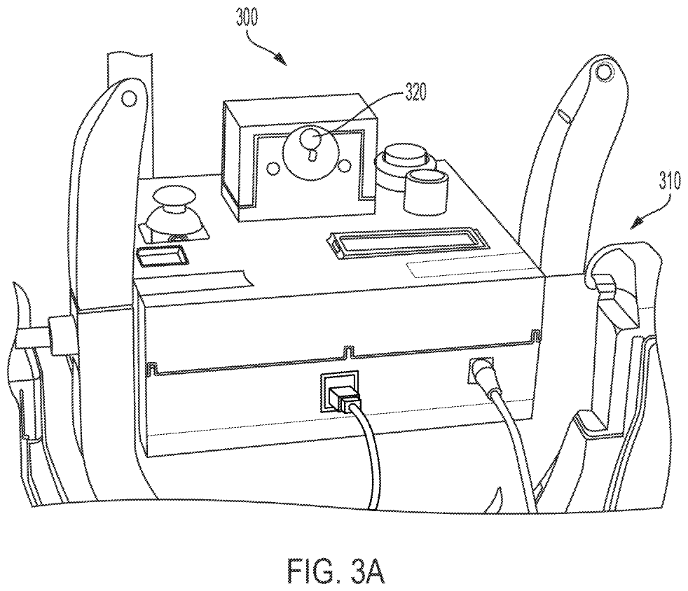

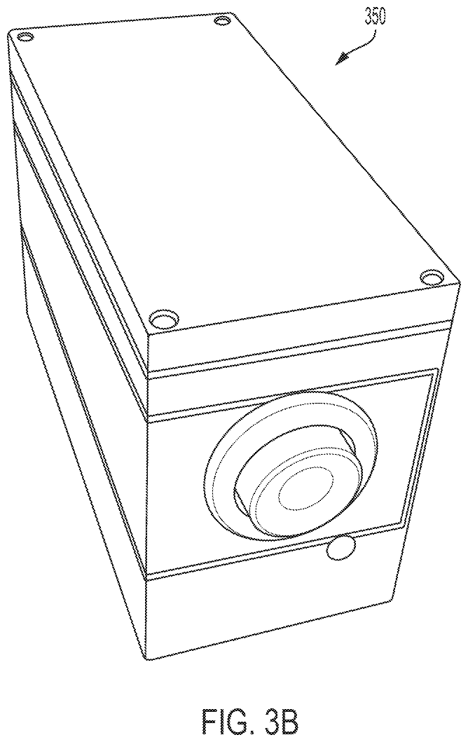

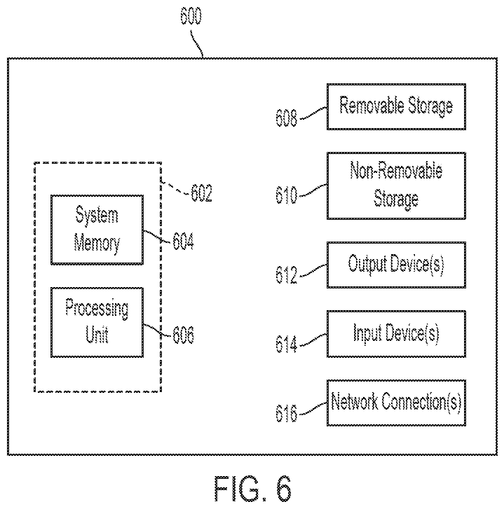

It should be understood that in some implementations the controller is separate from the appliance 100 and in communication with the appliance, and that in other implementations the controller can be located on or in the appliance (for example, inside the housing 102). An example controller 350 is illustrated in FIG. 3B. For example, the system can include a communication link that operably connects the controller and the appliance. An example implementation where the controller and appliance are separate and connected by a communication link is described herein with respect to FIG. 3A and FIG. 3B. The communications link can be a wired or wireless communications link. It should be understood that both the appliance 100 and controller (not shown) can include one or more computing devices (e.g., the computing device shown in FIG. 6) and that network connections 616 of the computing device 600 shown in FIG. 6 can be used to implement any combination of wireless or wired communications links.

The controller can include a processor and memory, as well as any or all of the components of the computing device 600 shown in FIG. 6.

The controller can be configured to control the appliance to implement a test sequence for quality assurance of a radiosurgery system. The test sequency can include transmitting one or more control signals to the appliance according to a QA protocol. The QA protocol can include moving the actuator from a first position to a second position, and thereby moving the attached indicator. The first position and second position can be separated by a predetermined distance.

In some implementations, the QA test protocol can include repeatedly moving the actuator between the first and second positions a predetermined number of times. Alternatively or additionally, the QA test protocol can include maintaining the actuator in the second position for a predetermined period of time, or maintaining the actuator in the first position for a predetermined period of time. For example, the QA protocol can include moving between the first position and second position a certain number of times, and maintaining the actuator in the first position for a first predetermined time period, and maintaining the actuator in the second position for a second predetermined time period.

Additionally, it should be understood that any number of positions of actuator are contemplated by the present disclosure. For example, in some implementations the QA protocol can include a third actuator position, and the QA protocol can include moving the actuator between the first, second, and third actuator positions.

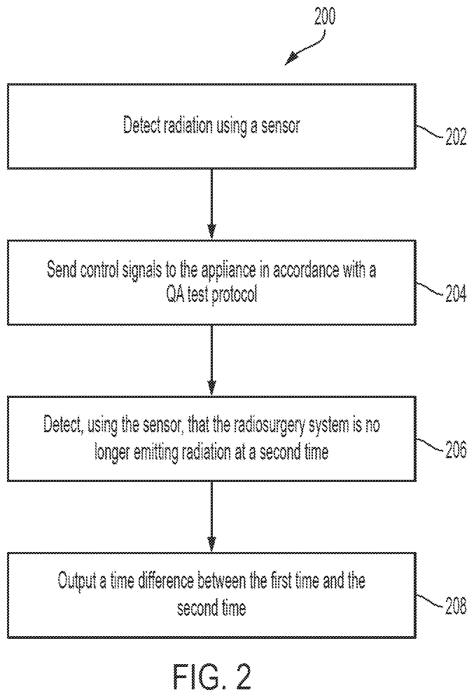

With reference to FIG. 2, an flowchart of an example test sequence 200 is shown. It should be understood that any or all of the operations of the test sequence illustrated in FIG. 2 can be performed separately, and in any order.

At block 202, the system can detect radiation from a radiosurgery system using a radiation sensor. At block 204, the system can send a control signal to the appliance in accordance with the QA test protocol at a first time.

As an example, the radiosurgery system can be configured to turn off the radiation beam or beams when the amount of movement of the indicator is greater than a certain predetermined amount. The QA protocol can be a protocol configured to determine whether the radiosurgery system actually stops emitting radiation when the indicator moves in an amount greater than the predetermined amount. The control signal can cause the actuator of the appliance to move the indicator a distance that is greater than the predetermined amount to check whether the radiosurgery system turns off the radiation beam, and to measure how long it takes for the beam to turn off.

At block 206, the system detects, using the radiation sensor, that the radiosurgery system is no longer emitting radiation at a second time. This can validate that the radiosurgery system is turning off in response to the indicator moving the predetermined distance.

At block 208, the system outputs a time difference between the first time and the second time. The time difference can be relevant to the safety of the system because if a patient moves while being treated, the system should turn off quickly to avoid delivering excess radiation to tissue outside the target area.

FIGS. 3A and 3B show an experimental implementation of the present disclosure. FIG. 3A illustrates the experimental implementation of an appliance 300 mounted to a radiosurgery system 310. The indicator 320 is shown. FIG. 3B illustrates a controller 350 that can be used to remote-control the appliance 300.

In the experimental implementation, the controller 350 can be located outside the treatment vault and connected to the main appliance 300 by an ethernet cable, has a single button and power on light. When the appliance 300 is connected to a power source, and the controller 350 is connected to the main box, pressing the button on the controller will cause the disc to rotate from its original position to a new position. This moves the indicator 320 being tracked by the HDMM system outside the tolerance threshold, which will cause the radiosurgery system to automatically pause the treatment when the device is inside the Gamma Knife bore and the sectors are open. Pressing the button again rotates the disc and indicator 320 back to its original position, and the HDMM system will automatically resume the treatment. If the indicator does not return to its original position in a certain amount of time, or the disc leaves the tolerance threshold a sixth time, the HDMM system will automatically interrupt the treatment and bring the couch and main box out of the bore. Additional features of the example system include a button on the main box that toggles the distance the disc will travel when the button on the controller is pushed, a joystick on the main box for manually rotating the disc, and a fiducial attachment that can be used if CBCT registration is required prior to treatment.

The experimental implementation was tested by running mask-based Gamma Knife treatments on this QA device. It satisfies the requirement of the QA check for the HDMM system. The device has also been calibrated such that movement distance indicated on the LED screen matches the amount of movement the IR dot, which is indicated by the HDMM system.

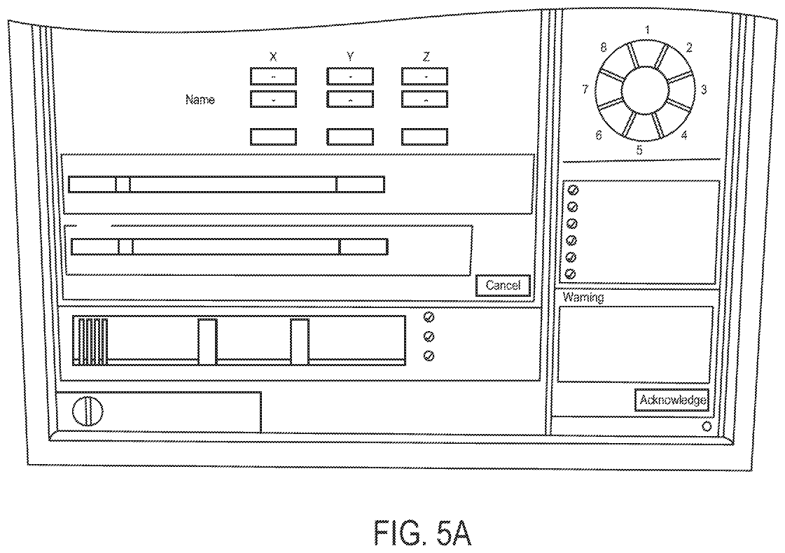

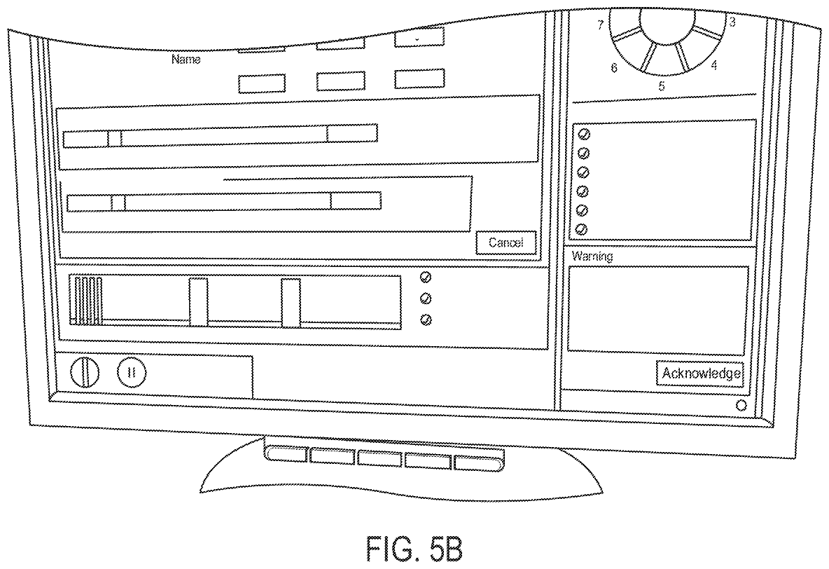

FIG. 4 illustrates the appliance 300 shown in a gamma knife system 400. FIG. 5A and FIG. 5B illustrate example illustrations experimental data. FIG. 5A illustrates test results where a patient has deviated from the treatment position a sixth time, after repeatedly deviated from the treatment position beyond a tolerance value for five times, and FIG. 5B illustrates test results where the patient has deviated beyond the tolerance from the treatment position continuously for more than 25 seconds.

It should be appreciated that the logical operations described herein with respect to the various figures may be implemented (1) as a sequence of computer implemented acts or program modules (i.e., software) running on a computing device (e.g., the computing device described in FIG. 6), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 6, an example computing device 600 upon which the methods described herein may be implemented is illustrated. It should be understood that the example computing device 600 is only one example of a suitable computing environment upon which the methods described herein may be implemented. Optionally, the computing device 600 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 600 typically includes at least one processing unit 606 and system memory 604. Depending on the exact configuration and type of computing device, system memory 604 may be volatile (such as random access memory (RAM), non-volatile (such as read-only memory (ROM), flash memory, etc.), or some combination of the two. This most basic configuration is illustrated in FIG. 6 by dashed line 602. The processing unit 606 may be a standard programmable processor that performs arithmetic and logic operations necessary for operation of the computing device 600. The computing device 600 may also include a bus or other communication mechanism for communicating information among various components of the computing device 600.

Computing device 600 may have additional features/functionality. For example, computing device 600 may include additional storage such as removable storage 608 and non-removable storage 610 including, but not limited to, magnetic or optical disks or tapes. Computing device 600 may also contain network connection(s) 616 that allow the device to communicate with other devices. Computing device 600 may also have input device(s) 614 such as a keyboard, mouse, touch screen, etc. Output device(s) 612 such as a display, speakers, printer, etc. may also be included. The additional devices may be connected to the bus in order to facilitate communication of data among the components of the computing device 600. All these devices are well known in the art and need not be discussed at length here.

The processing unit 606 may be configured to execute program code encoded in tangible, computer-readable media. Tangible, computer-readable media refers to any media that is capable of providing data that causes the computing device 600 (i.e., a machine) to operate in a particular fashion. Various computer-readable media may be utilized to provide instructions to the processing unit 606 for execution. Example tangible, computer-readable media may include, but is not limited to, volatile media, non-volatile media, removable media and non-removable media implemented in any method or technology for storage of information such as computer readable instructions, data structures, program modules or other data. System memory 604, removable storage 608, and non-removable storage 610 are all examples of tangible, computer storage media.

Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 606 may execute program code stored in the system memory 604. For example, the bus may carry data to the system memory 604, from which the processing unit 606 receives and executes instructions. The data received by the system memory 604 may optionally be stored on the removable storage 608 or the non-removable storage 610 before or after execution by the processing unit 606.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A quality assurance (QA) system for a radiosurgery system, the system comprising:
an appliance comprising:
a housing,
an indicator; and
an actuator configured to move the indicator;
a controller in operable communication with the appliance, wherein the controller comprises a processor and a memory, the memory having computer-executable instructions stored thereon that, when executed by the processor, cause the processor to:
transmit one or more control signals to the appliance in accordance with a QA test protocol, wherein the QA test protocol comprises moving the actuator from a first position to a second position, the first and second positions being separated by at least a first predetermined distance.

2. The system of claim 1, wherein the QA test protocol further comprises repeatedly moving the actuator between the first and second positions a predetermined number of times.

3. The system of claim 1 or claim 2, wherein the QA test protocol further comprises maintaining the actuator in the second position for a predetermined period of time.

4. The system of any one of claims 1-3, further comprising a communication link, wherein the communication link operably connects the controller and the appliance.

5. The system of claim 4, wherein the communication link is a wired communication link.

6. The system of claim 4, wherein the communication link is a wireless communication link.

7. The system of any one of claims 1-6, wherein the one or more control signals are configured to move the actuator a second predetermined distance.

8. The system of any one of claims 1-7, wherein the appliance further comprises a sensor configured to detect radiation.

9. The system of claim 8, wherein the sensor is a photodiode.

10. The system of claim 8, wherein the sensor is a ionization detector, scintillation detector or semiconductor detector.

11. The system of any one of claims 8-10, wherein the memory has further computer executable instructions stored thereon that, when executed by the processor, cause the processor to:
detect, using the sensor, a radiation emitted from the radiosurgery system;
while the radiation is detected by the sensor, send the one or more control signals to the appliance in accordance with the QA test protocol at a first time;
detect, using the sensor, that the radiosurgery system is no longer emitting the radiation at a second time; and
output a time difference between the first time and the second time.

12. The system of any one of claims 1-11, further comprising a fiducial marker.

13. The system of claim 12, wherein the fiducial marker is a removable fiducial marker.

14. The system of any one of claims 1-11, further comprising a plurality of fiducial markers comprising a first fiducial marker and a second fiducial marker.

15. The system of claim 14, wherein the first and second fiducial markers are offset from one another by a predetermined orientation and a predetermined position.

16. The system of claim 14 or claim 15, wherein the first fiducial marker is a removable fiducial marker.

17. The system of any one of claims 14-16, wherein the memory has further computer executable instructions stored thereon that, when executed by the processor, cause the processor to:
receive location information from the radiosurgery system, the location information comprising an estimate of the position of the first fiducial marker and an estimate of the position of the second fiducial marker, and
determine, based on the location information, an accuracy value for the radiosurgery system.

18. The system of any one of claims 1-17, wherein the appliance is operably coupled to the radiosurgery system.

19. The system of claim 18, wherein the memory has further computer executable instructions stored thereon that, when executed by the processor, cause the processor to:
receive an instruction from the radiosurgery system; and
control the actuator in response to the received instruction.

20. The system of any one of claims 18-19, wherein the memory has further computer executable instructions stored thereon that, when executed by the processor, cause the processor to:
transmit calibration information to the radiosurgery system.

21. The system of any one of claims 1-20, wherein the actuator is a linear actuator.

22. The system of any one of claims 1-20, wherein the actuator is a stepper motor.

23. The system of any one of claims 1-22, wherein the radiosurgery system comprises a linear accelerator (LINAC).

24. The system of any one of claims 1-23, wherein the radiosurgery system is a gamma knife system.

25. The system of any one of claims 1-24, wherein the indicator comprises a reflector.
